(19) United States
(12) Patent Application Publication
(10) Pub. No.: US 2025/0367369 A1
(43) Pub. Date: Dec. 4, 2025

(54) SYSTEM FOR INDUCTIVE CHARGING OF INFUSION PUMP

(71) Applicant: Tandem Diabetes Care, Inc., San Diego, CA (US)

(72) Inventors: Reza Farshchi, San Diego, CA (US); Michael Fitzgibbons, San Diego, CA (US); Maxwell Hume, San Diego, CA (US); Payman Hassabi, San Diego, CA (US); John Nadworny, San Diego, CA (US); William Trevaskis, San Diego, CA (US)

(21) Appl. No.: 19/225,150

(22) Filed: Jun. 2, 2025

Related U.S. Application Data
(60) Provisional application No. 63/654,225, filed on May 31, 2024.
(60) Provisional application No. 63/664,810, filed on Jun. 27, 2024.

(51) Int. Cl. A61M 5/142

(52) U.S. Cl. CPC A61M 5/14248; A61M 2205/583; A61M 2205/8206; A61M 2205/8243

(57) ABSTRACT

Embodiments of the present disclosure relate to inductive charging of an ambulatory infusion pump. A cover can be provided that can be selectively engaged with an inductive charging device. A cutout can be formed through the cover for receiving the ambulatory infusion pump and maintaining the pump in a position on the inductive charging device for optimal power transfer.

### PRIORITY CLAIM

The present application claims priority to U.S. Provisional Application No. 63/654,225 filed May 31, 2024 and to U.S. Provisional Application No. 63/664,810 filed Jun. 27, 2024, each of which is hereby incorporated by reference herein in its entirety.

### TECHNICAL FIELD

The present disclosure relates to medical pumps for delivering medicament to a patient and, more specifically, to a user-wearable pump.

### BACKGROUND

There are many applications in academic, industrial, and medical fields that benefit from devices and methods that are capable of accurately and controllably delivering fluids, such as liquids and gases, that have a beneficial effect when administered in known and controlled quantities. Such devices and methods can be particularly useful in the medical field where treatments for many patients include the administration of a known amount of a substance at predetermined intervals.

One category of such fluid delivery devices includes insulin injecting pumps developed for administering insulin to patients afflicted with Type 1 or Type 2 diabetes. Some insulin injecting pumps are configured as portable or ambulatory infusion devices that can provide continuous subcutaneous insulin injection and/or infusion therapy as an alternative to multiple daily injections of insulin via a syringe or an insulin pen. Such pumps can be worn or carried by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

One type of pump that has been developed is a patch pump, or micro pump. Patch pumps generally are small pumps that are carried directly on the skin under the user's clothing. Many such pumps are situated directly on the infusion site such that no tubing is required to deliver the insulin and/or other medicament to the patient. Other patch pumps can be positioned on the patient's body with a short length of tubing extending to a nearby infusion site. Not unlike other types of pumps, but perhaps more typically, patch pumps can be at least in part disposable, meant to be worn for a period of time such as, e.g., a day or two, and then discarded and replaced by a new patch pump. Other patch pump designs contemplate a disposable component, such as a cartridge that contains medicament, and a reusable or durable component. In such configurations, the disposable and durable components may be joined together by the patient or caregiver in preparation for delivery of the medicament. Still other patch pumps may include a rechargeable durable component as well as a refillable and reusable medicament cartridge.

Many ambulatory infusion pumps are powered by batteries that need to periodically be recharged. This is typically accomplished by inserting a connector of a cable into a port on the infusion pump (such as, e.g., a USB or similar port) that facilitates the transfer of power and/or data and plugging the other end of the cable into a wall outlet or other power source. Recently, a number of consumer devices have been developed that utilize electromagnetic induction to charge an internal battery of a device without the need for additional physical electrical connections. In such systems, a transmitting induction coil within a charging base creates an alternating electromagnetic field. When a receiving induction coil in the device is aligned with the transmitting induction coil, power is transferred to the device where it is used to charge the battery. Embodiments of portable infusion pumps that utilize inductive charging are disclosed in U.S. Pat. Nos. 9,993,595, 10,357,603, 11,305,057 and 11,872,368, each of which is hereby incorporated herein by reference in its entirety.

### SUMMARY

Embodiments of the present disclosure relate to wireless charging of an ambulatory infusion pump, such as by inductive charging. A cover can be provided that can be selectively engaged with an inductive charging device. A cutout can be formed through the cover for receiving the ambulatory infusion pump and maintaining the pump in a position on the inductive charging device for optimal power transfer.

In an embodiment, a cover for an inductive charging device for an ambulatory infusion pump includes an upper surface having a cutout therethrough, the cutout defining an inner perimeter having a shape generally matching a shape of an outer perimeter of an ambulatory infusion pump. A rim can extend downwardly from the upper surface and be configured to engage an outer perimeter of an inductive charging device. A clip can extend downwardly from the rim and be configured to releasably engage with the inductive charging device.

An ambulatory infusion pump system including an ambulatory infusion pump including a rechargeable battery and an inductive charging device configured to recharge the rechargeable battery of the ambulatory infusion pump when the ambulatory infusion pump is placed on the inductive charging device. A cover can be selectively attachable to the inductive charging device. The cover can include an upper surface having a cutout therethrough, the cutout defining an inner perimeter having a shape generally matching a shape of an outer perimeter of the ambulatory infusion pump. A rim can extend downwardly from the upper surface and be configured to engage an outer perimeter of the inductive charging device. A clip can extend downwardly from the rim and be configured to releasably engage with the inductive charging device.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict an ambulatory infusion pump with an infusion set according to an embodiment of the disclosure.

FIGS. 2A-2B depict an ambulatory infusion pump according to an embodiment of the disclosure.

FIGS. 3A-3C depict an inductive charging device according to an embodiment of the disclosure.

FIGS. 4A-4F depict a cover for use with an inductive charging device according to an embodiment of the disclosure.

FIGS. 5A-5D depict the cover of FIGS. 4A-4F installed on an inductive charging device according to the embodiment of the disclosure.

FIGS. 6A-6D depict the cover and inductive charging device of FIGS. 5A-5D with a pump disposed thereon according to an embodiment of the disclosure.

FIGS. 7A-7C depict the cover and inductive charging device of FIGS. 5A-5D with a pump disposed thereon according to an embodiment of the disclosure.

FIGS. 8A-8E depict a cover for use with an inductive charging device according to an embodiment of the disclosure.

FIG. 9 depicts a flowchart of steps in a method of charging an ambulatory infusion pump according to an embodiment of the disclosure.

FIGS. 10A-10E depict a cover for use with an inductive charging according to an embodiment of the disclosure.

FIGS. 11A-11B depict the cover of FIGS. 10A-10E on an inductive charging device with a pump disposed therein according to an embodiment of the disclosure.

FIGS. 12A-12B depict the cover of FIGS. 10A-10E on an inductive charging device with a pump disposed therein according to an embodiment of the disclosure.

FIGS. 13A-13C depict a cover for use with an inductive charging device according to an embodiment of the disclosure.

FIGS. 14A-14C depict a cover for use with an inductive charging device according to an embodiment of the disclosure.

FIGS. 15A-15C depict a cover for use with an inductive charging device according to an embodiment of the disclosure.

FIGS. 16A-16C depict a cover for use with an inductive charging device according to an embodiment of the disclosure.

FIGS. 17A-17C depict a cover for use with an inductive charging device according to an embodiment of the disclosure.

FIGS. 18A-18C depict a cover for use with an inductive charging device according to an embodiment of the disclosure.

FIGS. 19A-19C depict a cover for use with an inductive charging device according to an embodiment of the disclosure.

FIGS. 20A-20C depict a cover for use with an inductive charging device according to an embodiment of the disclosure.

FIG. 21 depicts a cover for use with an inductive charging device according to an embodiment of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

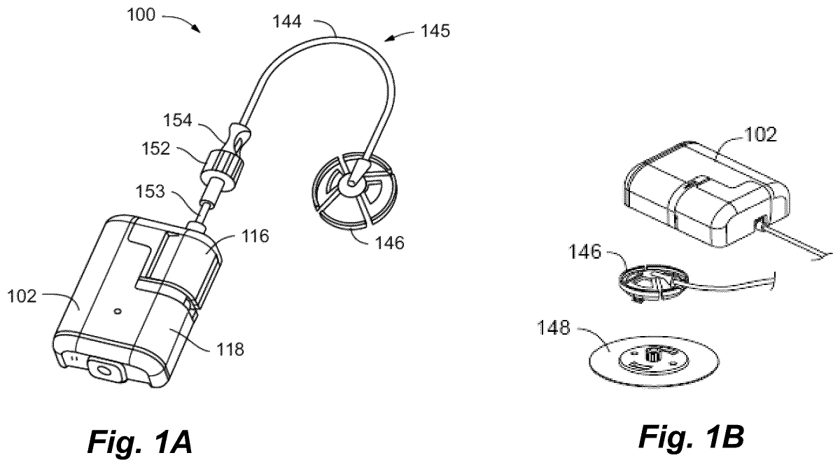

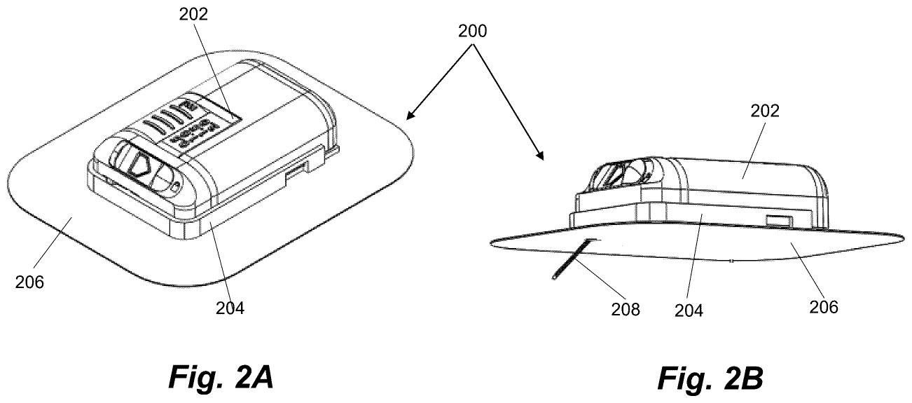

FIGS. 1A-1B and 2A-2B depict exemplary medical devices that can be used with embodiments of the disclosure. In this embodiment, the medical devices are configured as a pump 102, 202, such as an infusion pump, that can include a pumping or delivery mechanism and a reservoir for delivering a medicament to a patient. In one embodiment, the medical device can be a portable pump configured to deliver insulin to a patient. Further details regarding such pump devices can be found in U.S. Pat. Nos. 8,287,495, 10,279,107 and 10,864,318, each of which is incorporated herein by reference in its entirety. In other embodiments, the medical device can be an infusion pump configured to deliver one or more additional or other medicaments to a patient.

As depicted in FIGS. 1A-1B, pump system 100 can include a pump 102 and an infusion set 145. In embodiments, pump system 100 can include a drive unit 118 and a cartridge 116 having a short length of tubing 153 and a connector 152 extending therefrom. Infusion set 145 can include tubing 144 extending between a connector 154 and a site connector 146. Connector 154 on infusion set 145 can be configured to couple to pump 102 at connector 152 of pump 102. As shown in FIG. 1B, site connector 146 can be configured to be attached to an infusion hub 148 at an infusion site on a user through which medicament from the pump is delivered to a patient through a cannula extending from the infusion hub 148 into the user's skin.

FIGS. 2A-2B depict an infusion pump system 200 according to another embodiment of the disclosure. System 200 can include an infusion pump 202 configured as a patch pump that is worn directly on the body of the user with an adhesive patch 206. Pump 202 can be affixed to the body of the user with an adhesive patch 206 carrying a tray 204 thereon that releasably attaches the pump 202 to the tray 204. Pump 202 can be configured to deliver medicament to the user through a cannula 208 extending directly beneath the pump 202 and adhesive patch 206.

In embodiments, pumps 102, 202 include a processor that controls the operations of the pump and may communicate in either one-way or two-way modes to, e.g., receive operational commands and/or other signals, including data, from a separate device and/or, e.g., to send signals, including data to a separate device. Pumps 102, 202 can include one or more buttons configured to cause the processor to initiate one or more functions. Pumps 102, 202 can include only a single button although more than one button may be present on pump. Button can be configured to, for example, initiate delivery of medicament (e.g., a “quick bolus”). Any single button can be utilized to execute a plurality of functions or operations. For example, a single press of button may initiate one function, holding the button down for a predetermined period of time may initiate another function, etc. Because the depicted pumps 102, 202 optionally do not include a display or user interface, information and feedback regarding medicament delivery or dosing or other functions initiated with button can be communicated to and displayed on a remote control device or other device having a display and/or other type of user interface.

In embodiments, pumps 102, 202 can include a light source, such as a light emitting diode (LED). Light source can be configured to provide user feedback regarding user input and/or the performance of a desired function. For example, in one embodiment, light source can illuminate or blink one or more times to indicate that the one or more buttons have been activated and/or that a desired function has been initiated. In one embodiment, pump can additionally and/or alternatively vibrate and/or provide audible notifications to indicate that the one or more buttons have been activated and/or that a desired function has been initiated or, e.g., to provide user feedback regarding user input and/or the performance of the desired function. Illumination of light source and/or vibrations and/or audible notifications may be executed in any number of patterns, frequencies, durations, sequences, combinations, colors, brightness levels, etc. to indicate particular information, such as particular input received and/or particular functions or operations enabled and/or initiated, to the pump user or caregiver. In some embodiments, the pump includes two indicator lights or more indicator lights.

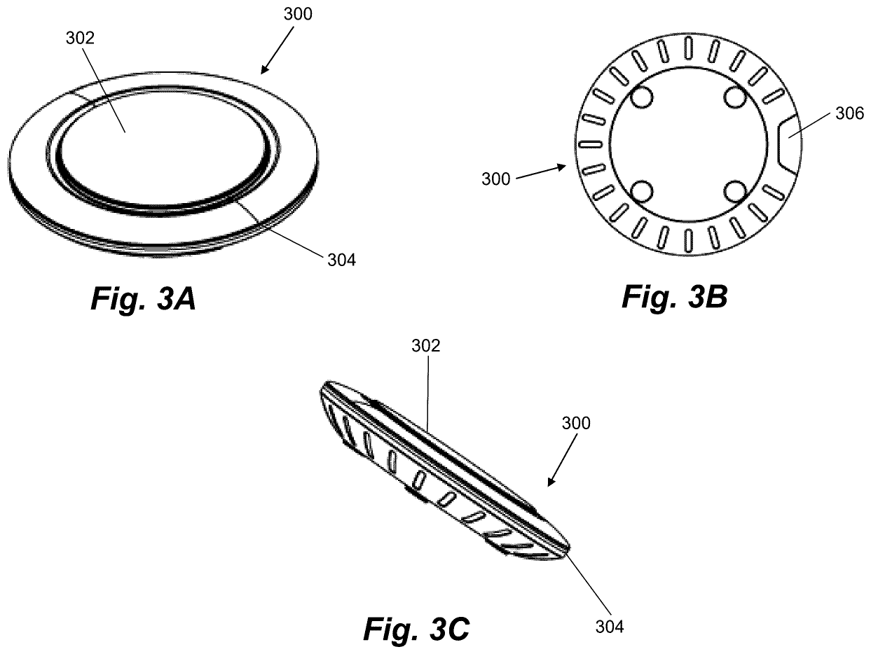

Pumps according to embodiments of the present disclosure can include one or more rechargeable batteries in and/or associated with the pump drive unit. In some embodiments, a rechargeable battery can be wirelessly charged, for example through inductive charging by an inductive charging pad such as the inductive charging pad 300 depicted in FIGS. 3A-3C. In some embodiments, the charging pad 300 may receive power by being connected to a wall outlet. In other embodiments, the charging pad 300 may additionally or alternatively include a wired and/or wireless power connection to, for example, a computer (e.g., via USB or IEEE 1394), a 12 volt automobile outlet, a battery pack (e.g., via USB or IEEE 1394), optical means, and/or a solar panel, among others. Charging pad 300 is exemplary and inductive charging devices of various other configurations can be employed to charge the batteries of the pumps disclosed herein.

Inductive charging of pump can be carried out according to known standards, such as, for example, the Qi standard. In such a system, both the pump and the charging pad 300 include an inductive coil. A transmitting coil in the charging pad 300 connected to the power source generates an oscillating magnetic field that induces an alternating current in a receiving coil in the pump to transfer power to pump when the pump is placed on the upper surface 302 of the charging pad. In one embodiment, charging pad 300 is continuously or periodically (e.g., twice a second) sending an analog signal to detect the presence of a pump. When the pad detects the presence of a pump (via a magnetic load), it sends a digital communication signal to, e.g., a processor associated with the pump. The pump processor receives the signal and directs and/or sends a return signal to the pad, which causes the pad to transmit charging power to the pump. Other types of inductive or other wireless charging can also be employed including, for example Near Field Communication (NFC) charging (which can also include data transfer between the pump and the device).

To charge a pump, a pump such as pump 102 employed with an infusion set 145 may first be disconnected from the tubing of the infusion set. The pump can then be placed onto the charging pad 300 where its battery will automatically be inductively (re) charged when the pad 300 is connected to a power source. In another embodiment, the pump 102 can remain connected to the infusion set and the user while the battery is recharged. A pump 202 carried on a pump holder 204 worn on a body of a user can be removed from the holder 204 and placed on the inductive charging pad. In some embodiments, a pump that can be inductively charged does not include a connection, such as a USB port, into which a power cord can be inserted for power transfer. Such an embodiment provides the advantages of being more robust for waterproofing because of the lack of exposed electrical contacts and obviating electrical isolation requirements imposed upon such connections.

The efficiency with which a pump charges can be increased and the time it takes to charge the pump decreased the more optimally that the receiving coil of the pump is positioned with respect to the transmitting coil of the inductive charging pad. Not only is initial positioning of the pump important, it is also important that the pump is not accidentally dislodged during charging or that vibrations during charging do not cause the pump to move on the charging pad.

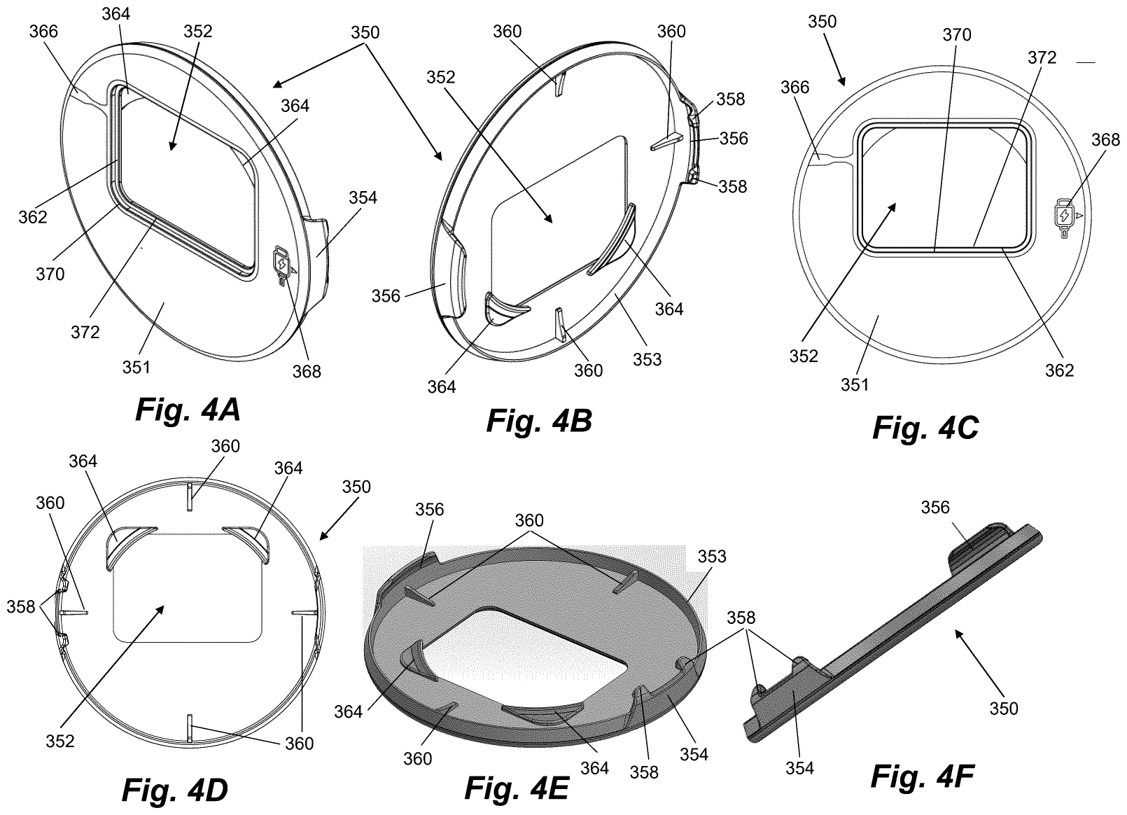

As such, referring now to FIGS. 4A-4F, in some embodiments a cover 350 can be provided for use with the charging pad 300 having a cutout 352 sized to receive a pump 102, 202 in order to properly position and retain the pump on the charging pad 300 during recharging formed through an upper surface 351 of charging pad 300. Cutout 352 can be formed in a location to position the pump for optimal charging for a particular charging pad 300. A ledge 362 can be provided around a perimeter of cutout 352. Ledge 362 essentially provides for cutouts of two different sizes to accommodate two different sizes of pump (e.g., a pump with and without a cover as will be discussed in more detail below). In particular, cutout 352 provides a first, larger inner perimeter 370 that extends from cutout 352 down to ledge 362. The inner perimeter surface of ledge 362 provides a second, smaller inner perimeter 372 beneath the first inner perimeter.

Cover 350 can include one or more clips 354, 356 that aid in positioning and/or retaining the cover on the charging pad. In embodiments, at least one clip 354 can include anti-rotation projections 358 that interface with the charging pad to prevent the cover 350 from rotating on the charging pad. The other clip 356 can be a flexible projection curved to conform to an outer perimeter 304 of the charging pad. Clip 356 can be deformed to fit over the pad and provide a snap fit with pad. Ramp features 360 can be provided on an underside of cover 350 that interface with curved surfaces of the charging pad to provide a stable, flush fit between cover 350 and charging pad 300. Cover 350 can further include tabs 364 on the underside of cover adjacent cutout 352. Tabs 364 conceal gaps on the charging pad's curved surface to provide a visually smooth appearance.

Cover 350 can also include one or more visual guide features. For example, cover 350 includes a pump orientation guide feature 366 shaped like infusion tubing to indicate to the user that a pump with infusion tubing should be aligned such that the tubing aligns with the guide feature. Aligning the pump in this orientation may provide more efficient power transfer than aligning the pump in the opposite orientation. A cover alignment guide feature 368 can indicate how the cover 350 should be oriented with that feature adjacent to the power port of the charging pad. As will be described in more detail below, aligning cover 350 in this orientation enables anti-rotation features 358 to be inserted into the recess 306 of the charging pad 300 containing the power port for the charging pad 300.

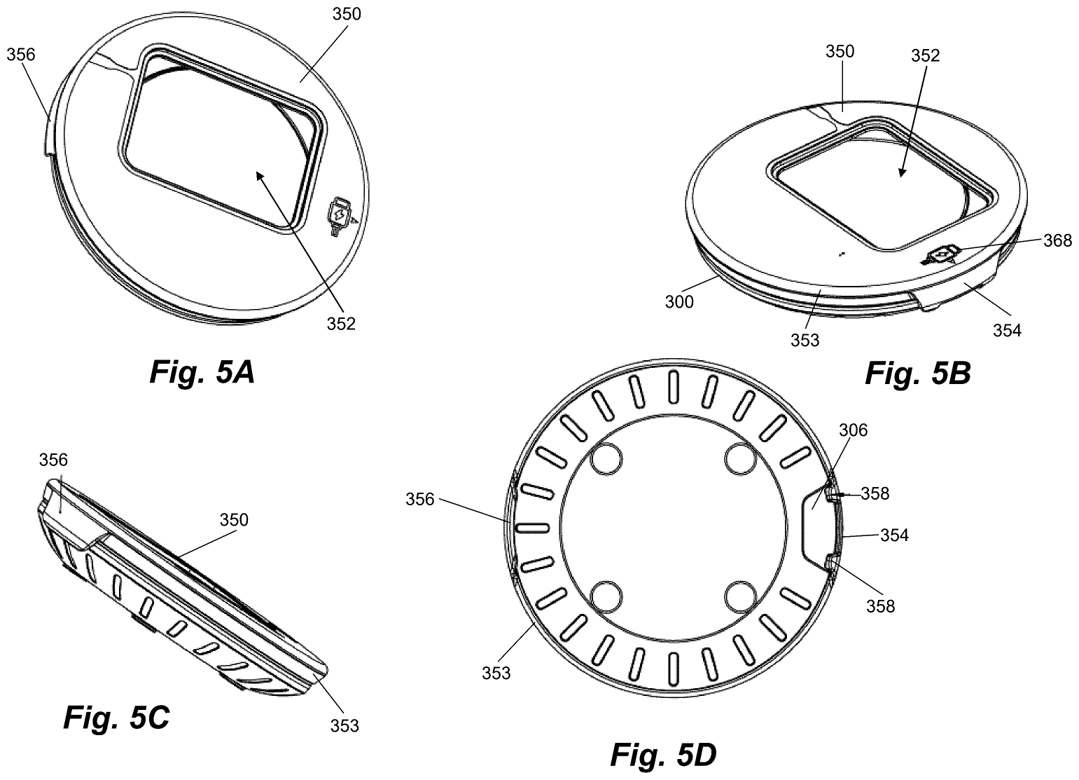

FIGS. 5A-5D depict the cover 350 of FIGS. 4A-4F installed on the charging pad of FIGS. 3A-3C. Top surface 351 of cover 350 covers the top of the charging pad 300, expect for the location of the cutout 352, and a downwardly extending rim 353 of cover 350 surrounds the outer perimeter 304 of the charging pad 300. As noted above, cover alignment guide feature 368 on the surface of the cover 350 can indicate the user the proper rotational alignment of the cover 350 on the charging pad 300. In particular, cover alignment guide feature 368 guides a user to position clip 354 adjacent the power port recess 306 on the underside of the charging pad 300. Clip 354 will flex outwardly over charging pad 300 while being placed thereon and then snap back when fully seated on the charging pad to nest the anti-rotation features 358 within the recess 306 to prevent rotation of the cover 350 on the charging pad. The opposing clip 356 will also flex outwardly and then conform to the outer perimeter of the charging pad to cooperate with clip 354 to securely retain the cover 350 on the charging pad. The cover 350 can be removed by flexing one or more of the clips 354, 356 outwardly to disengage the clips from the charging pad 300 and lifting the cover 350 upwardly off of the charging pad 300.

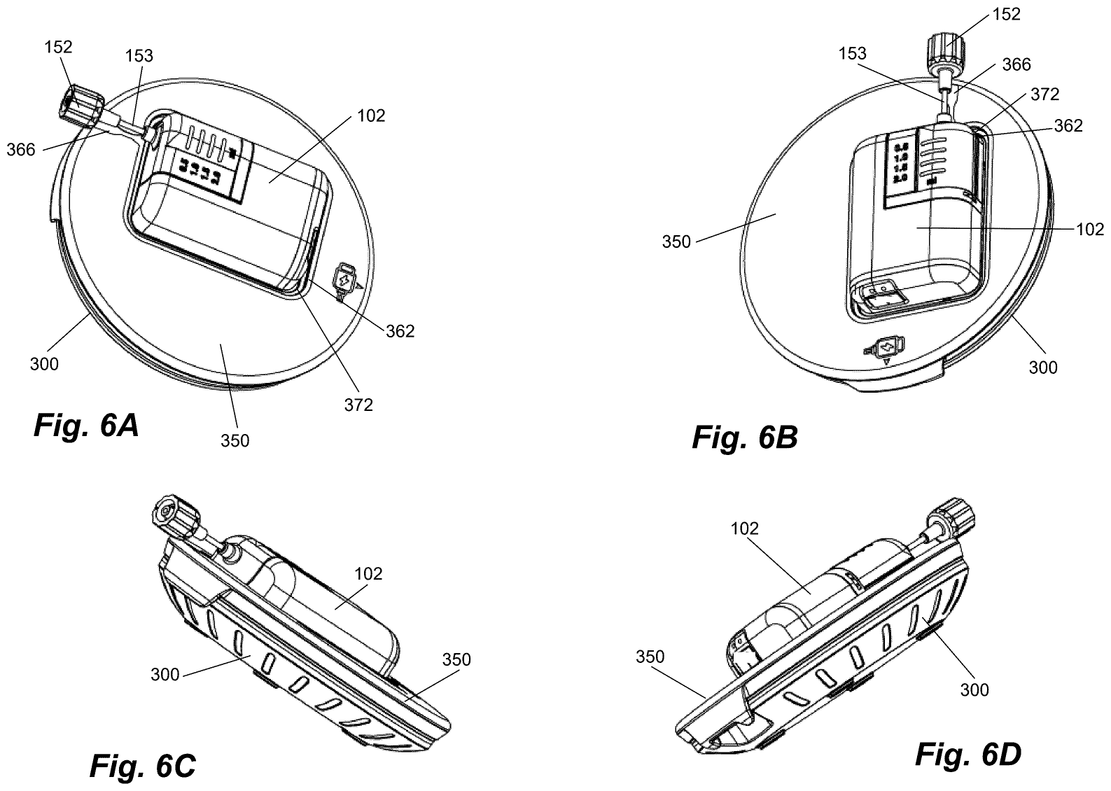

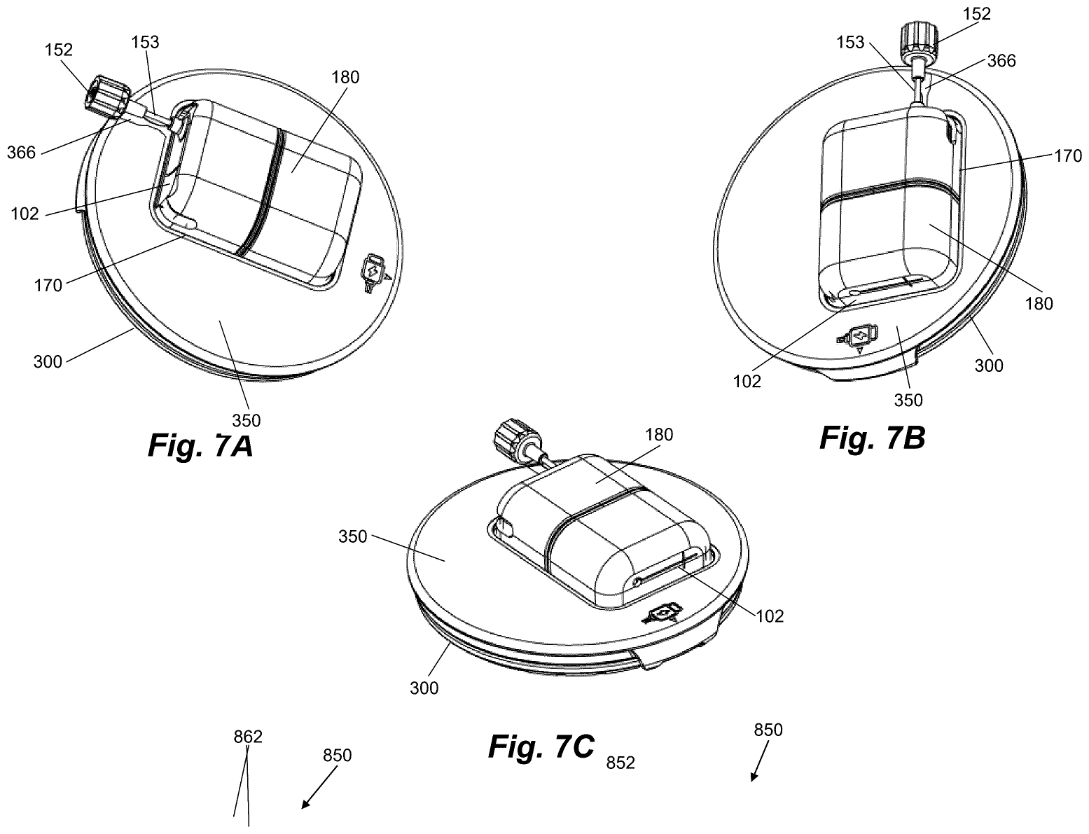

FIGS. 6A-6D and 7A-7C depict a pump 102 placed on an inductive charger 300 utilizing a cover 350 as described herein. Pump orientation guide feature 366 guides a user to orient the pump 102 with the infusion tubing 152 and connector 153 aligned along guide feature 366 for optimal charging. Alternatively, charger 300 and cover 350 can be utilized with a pump such as pump 202 that does not include tubing and a connector. In the embodiment of FIGS. 6A-6D, the pump 102 nests within the ledge 362 in cutout 352. That is, pump 102 rests directly on the top surface of the inductive charger 300 with the inner perimeter 372 of the ledge 362, which has a size and shape matching the size and shape of the outer perimeter of pump 102, surrounding the pump 102 and preventing movement of the pump along the surface of charger 300. FIGS. 7A-7C depict a pump 102 having a protective case 180 surrounding the pump 102. Protective case 180 enlarges the outer perimeter of pump 102. As such, in this configuration the pump 102 rests on top of the ledge 362 such that an inner perimeter of the cutout 352 above the ledge surrounds and maintains the positioning of the pump 102 on the charging pad 350 in the protective case. Although this means that the pump 102 will rest slightly above rather than directly on the surface of the charging pad 350, charging power will still be transmitted from the charging pad 350 to the pump 102. Any decrease in charging efficiency from this arrangement is balanced by the ability to charge the pump 102 without having to remove it from the protective case 180.

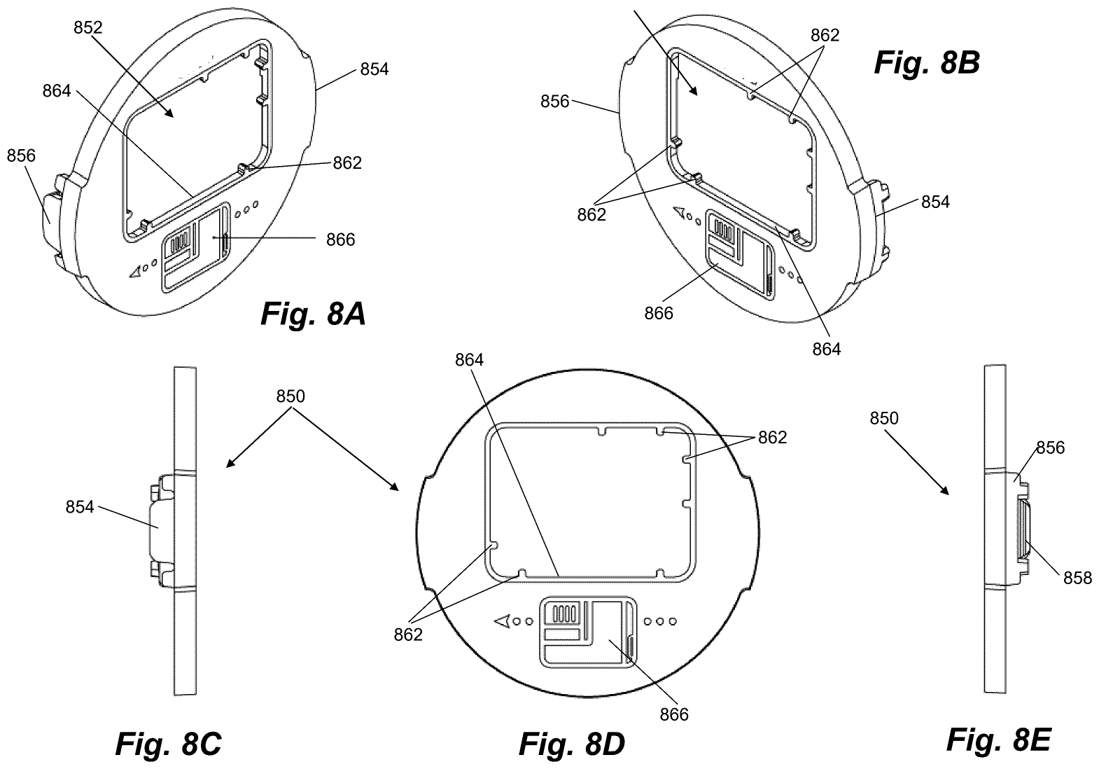

FIGS. 8A-8E depict a cover 850 for use with the inductive charging device of FIGS. 3A-3C according to another embodiment of the disclosure. Cover 850 includes a cutout 852 sized to receive an infusion pump to align the pump on a charging pad. One or more clips 854, 856 can releasably retain the cover 850 on the charging pad. In this embodiment, clip 856 includes a tab 858 that aids in flexing clip 856 outwardly for inserting the cover 850 onto and removing the cover 850 off of the charging pad. A pump orientation guide feature 866 can also be provided on cover 850 to guide the user as to how to orient the pump in the cutout 852. This embodiment is also designed to accommodate a pump with and without a protective cover (see cover 180 in FIGS. 7A-7C). Cutout 852 includes a plurality of protrusions 862 extending into the opening defined by cutout 852. Cutout 852 and protrusions 862 are sized such that when a pump without a protective case is placed within cutout, the protrusions 862 abut the outer perimeter of the pump to maintain the alignment of pump on the charging pad. Cover 850 is designed to accommodate a specially designed protective case that includes apertures in case through which protrusions 862 are inserted. When inserted into the cutout 852, the pump and protective case will abut an inner perimeter 864 of the cutout 852 with the protrusions 862 extending into the protective case.

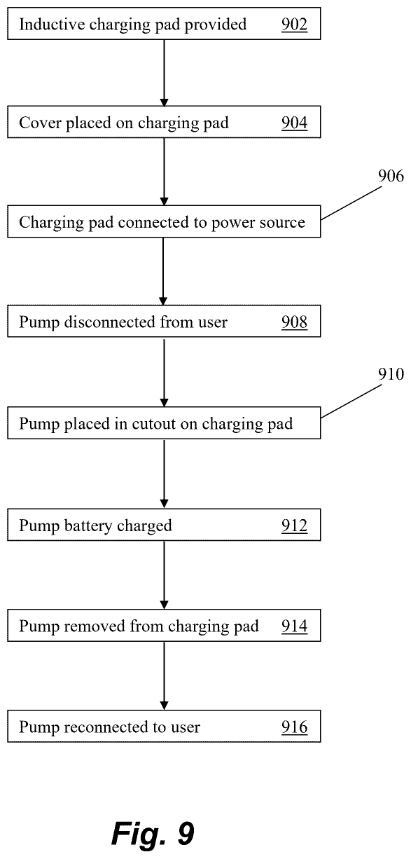

FIG. 9 depicts a flowchart of steps in a method 900 of charging an ambulatory infusion pump according to an embodiment of the disclosure. At step 902, an inductive charging pad is provided. A cover such as cover 350 or 850 having a cutout for receiving the pump can be placed onto the inductive charging pad at step 904. The inductive charging pad is connected to a power source at 906. The pump can be disconnected from the user's body at step 908. Although, these steps 902-908 are described as occurring in a particular order, it should be noted that these steps can be performed in any other order. For example, the pump may be disconnected from the body prior to the inductive charging pad being connected to a power source, the cover may be placed on the charging pad after connecting the charging pad to a power source, etc. Once these steps have been completed, at step 910 the pump can be placed in the cutout on the charging pad and can remain on the charging pad at step 912 to charge the battery to a desired level. Once the pump has been charged to the desired level, it can be removed from the charging pad at step 914 and reconnected to the user to resume delivery of medicament at step 916.

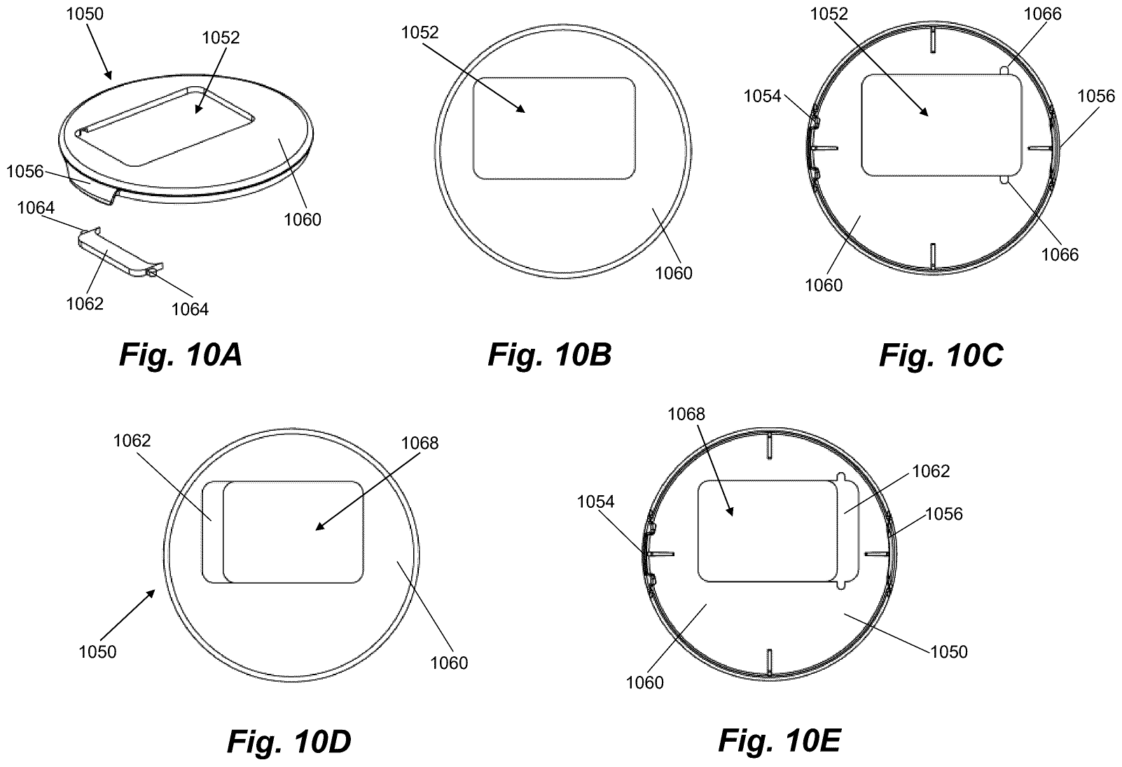

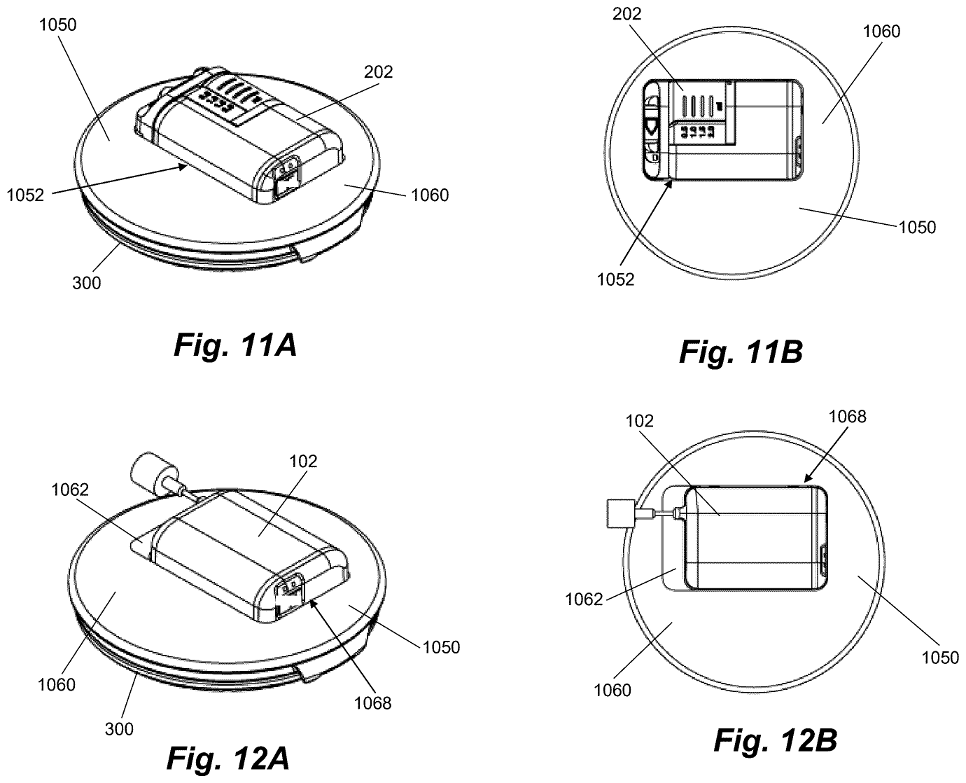

FIGS. 10A-10E depict a cover 1050 for use with an inductive charging device according to another embodiment of the disclosure. Cover 1050 is designed to accommodate pumps of varying sizes and can include a cutout 1052 and one or more clips 1054, 1056 (which may or may not be configured similarly to clips 354, 356) that aid in retaining cover 1050 on an inductive charging device. Cover 1050 comprises a body 1060 that includes cutout 1052 and clips 1054 and an insert 1062. Insert 1062 can be selectively attachable to body 1060 by inserting projections 1064 of insert 1062 into corresponding slots 1066 in body 1060. When insert 1062 is attached to body 1060, it effectively modifies the size of cutout 1052 to a smaller cutout 1068 to accommodate a pump of a different size. For example, FIGS. 11A-11B depict a pump 202 of a first, larger size inserted into cutout 1052 of cover 1050 without insert 1062 with the cutout 1052 conforming to the size and shape of the pump 202 to retain the pump 202 in optimal positioning on the charging pad 300. FIGS. 12A-12B depict a second, smaller pump 102 placed into cutout 1068 of cover 150 with insert 1062 inserted into body 1060 with the cutout 1068 conforming to the size and shape of the smaller pump 102. Cover 1050 including body 1060 and insert 1062 therefore provides a user with the versatility to optimally position and retain pumps having two different form factors on a given charger. Various other inserts and configurations can also be provided to accommodate other pumps as desired.

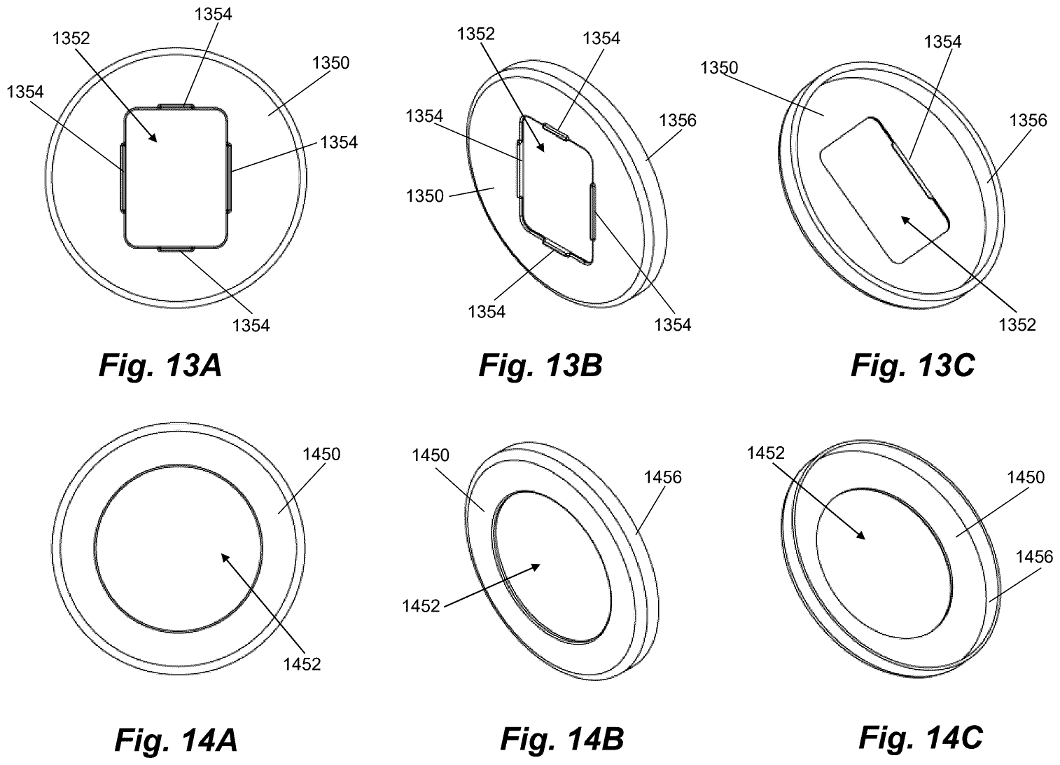

FIGS. 13A-13C, 14A-14C, 15A-15C, 16A-16C, 17A-17C, 18A-18C, 19A-19C and 20A-20C depict various additional embodiments of covers for use with an inductive charging device according to the disclosure.

FIGS. 13A-13C depict a cover 1350 having projections 1354 surrounding cutout 1352 for enhanced pump retention and a downwardly extending rim 1356 to retain the cover 1350 on the charging pad.

FIGS. 14A-14C depicts a cover 1450 having a larger cutout 1452 that doesn't conform to the shape of a particular pump and can therefore accommodate pumps of more varied shapes and sizes. Cover 1450 can be positioned on an inductive charging pad with downwardly extending rim 1456.

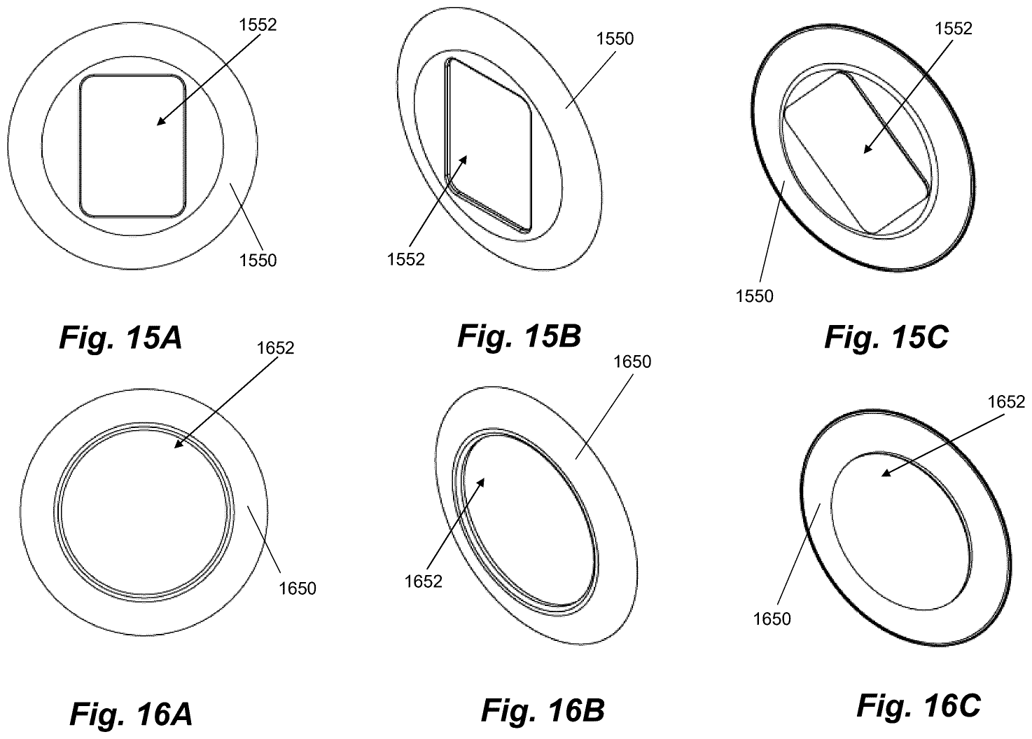

FIGS. 15A-15C depict a cover 1550 having a cutout 1552 sized to retain a pump therein that doesn't include any retention features for attaching the cover 1550 to an inductive charging pad. Cover 1550 can be glued or other adhered to be secured to a charging pad.

Cover 1650 depicted in FIGS. 16A-16C is similar to cover 1550 in that it is primarily designed to be glued or otherwise adhered to an inductive charging pad. Cover 1650 includes a larger cutout 1652 similar to FIGS. 14A-14C that can accommodate pumps of more varied shapes and sizes.

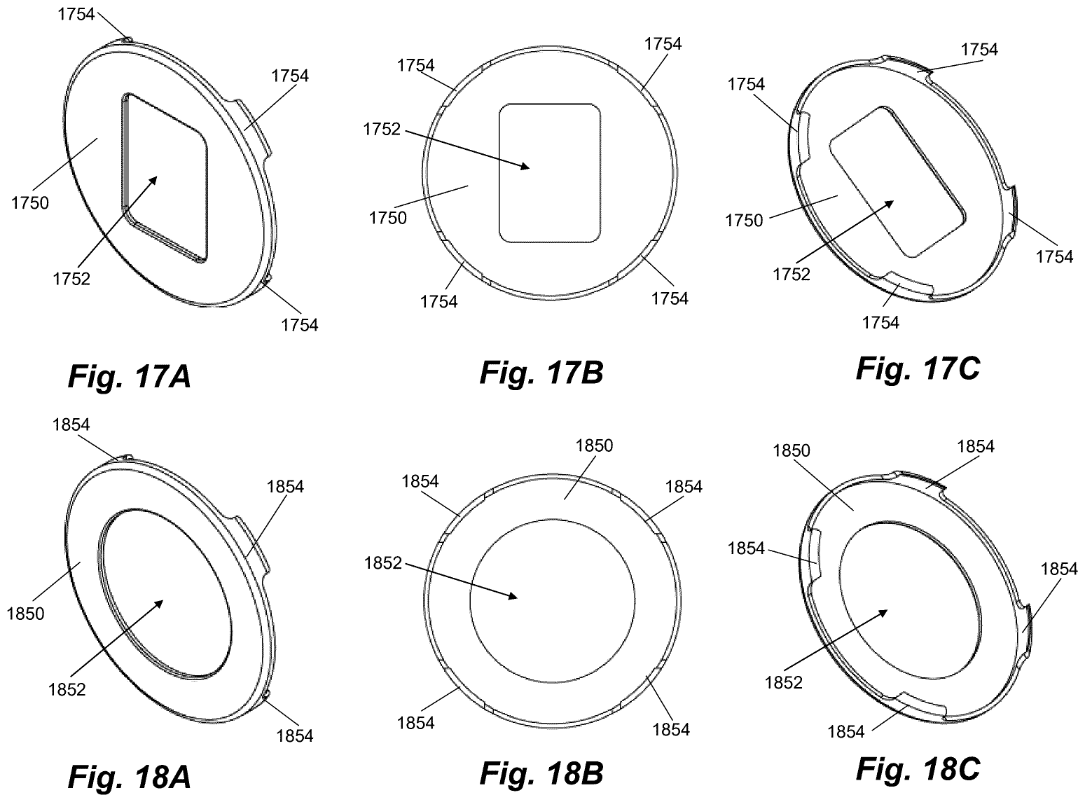

FIGS. 17A-17C and 18A-18C depict covers 1750, 1850 having four downwardly extending clips 1754, 1854 for securely attaching the covers 1750, 1850 to an inductive charging pad. Cover 1750 includes a cutout 1752 sized and shaped to correspond to a particular pump whereas cover 1850 has a cutout 1852 sized and shaped to accommodate pumps of varying sizes and shapes.

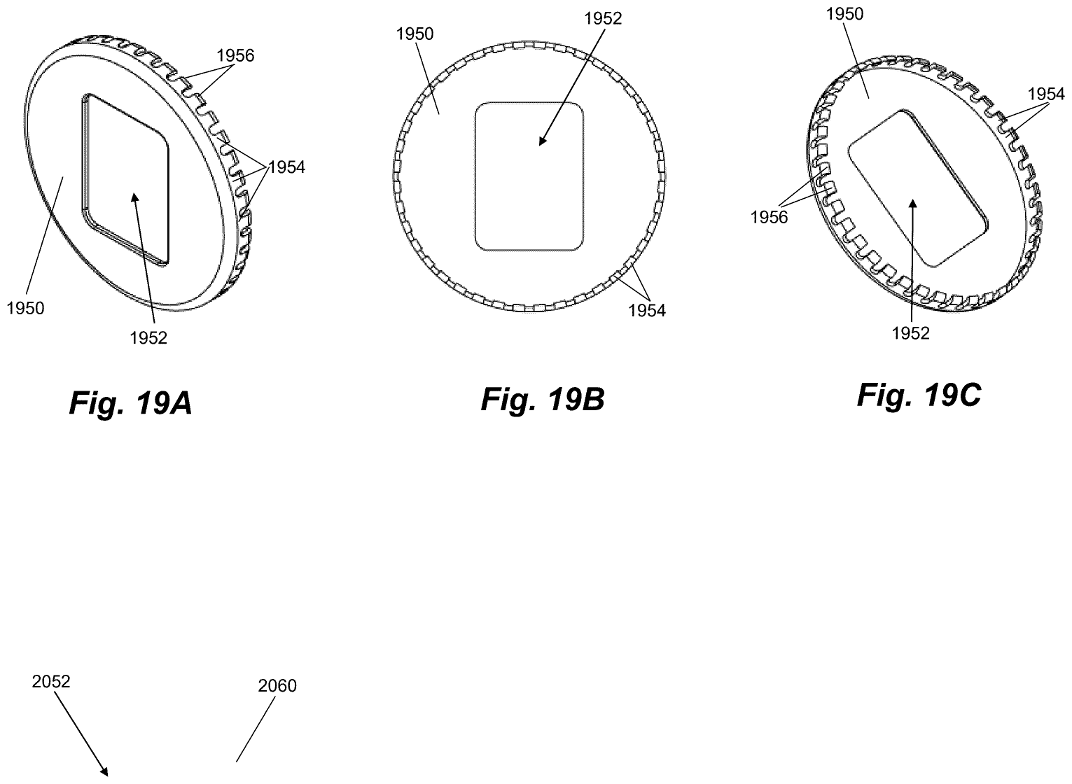

Cover 1950 depicted in FIGS. 19A-19C includes a plurality of repeating downward projections 1954 extending around the entire circumference of cover 1950. In contrast to the downwardly extending rim of certain embodiments (i.e., rim 1356 of cover 1350 in FIGS. 13A-13C), the gaps between projections can be aligned with lights on a charging pad such that the lights can remain visible when the cover 1950 is on the charging pad. Projections 1954 may have angled or curved tips 1956 configured to snap onto charging pad to firmly retain the cover 1950 on the pad. In the depicted embodiment, cutout 1952 is sized and shaped to accommodate a particular pump, but cutouts of various other sizes and shapes can be employed.

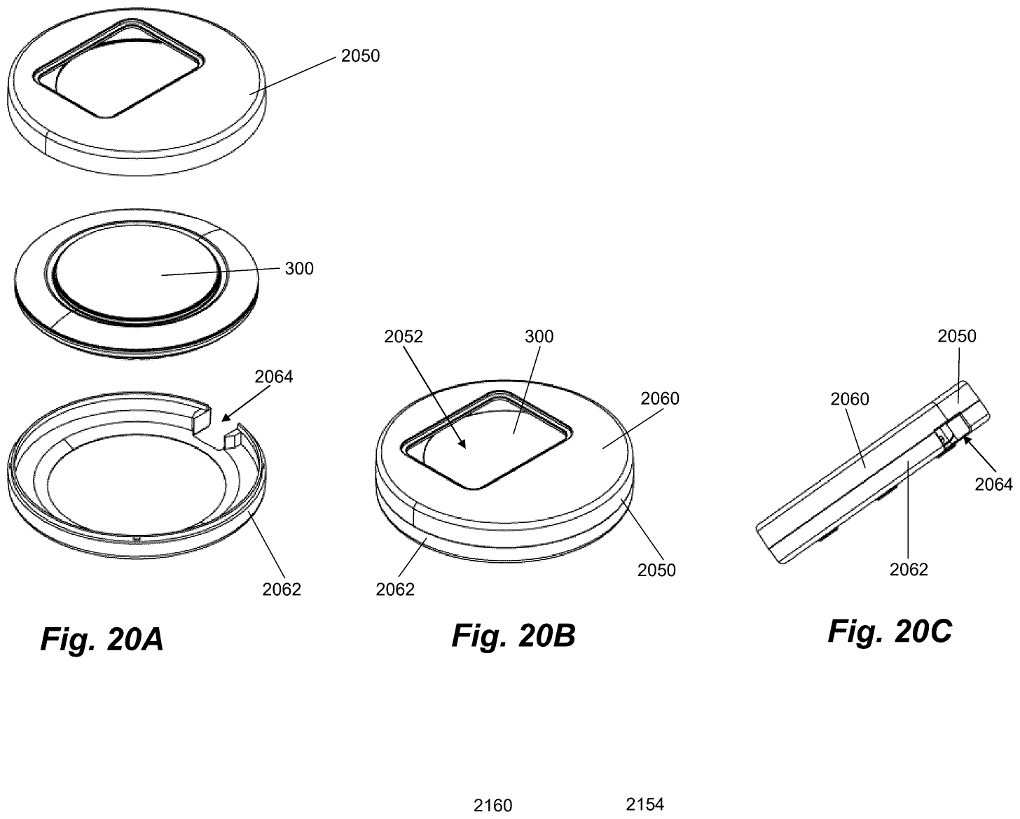

FIGS. 20A-20C depict a two-piece cover 2050 designed to fully encapsulate a charging pad 300. Cover 2050 can include a top portion 2060 and a bottom portion 2062 that interlock to contain the charging pad 300 therebetween. An aperture 2064 can be provided in bottom portion 2062 to provide access to a power port on charging pad 300 that enables access by a cord to connect charging pad 300 to a power source such as a wall outlet. The depicted cutout 2052 is similar to cutout 352 described above that accommodates a pump with and without a protective case, but cutouts of various other sizes and shapes can be employed.

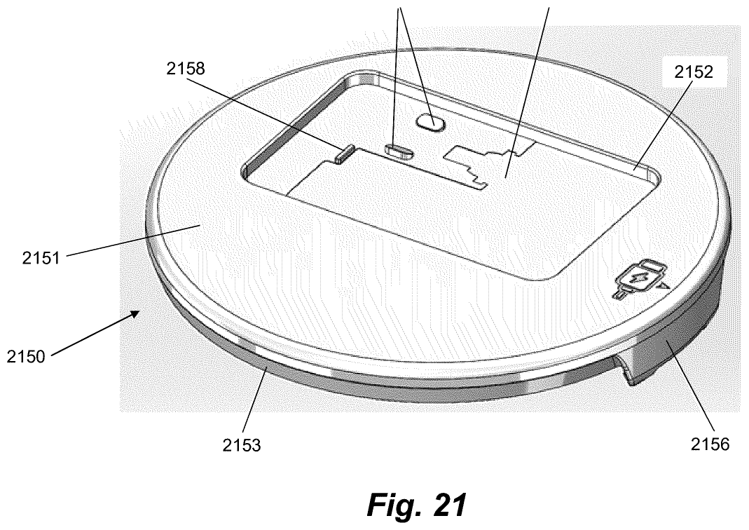

FIG. 21 depicts another cover 2150 for an inductive charging device according to the disclosure. Cover 2150 can include a cutout 2152 sized and shaped to receive a pump recessed into a top surface 2151 of cover 2150. In this embodiment, cutout 2152 does not extend all the way through the cover 2150 and instead defines a charging surface 2154 on which the pump will rest during charging within cutout 2152. A retention projection 2158 can extend upwardly from the charging surface 2154. Retention projection 2158 can nest within a corresponding recess on an underside of a pump where the cartridge and drive unit of the pump interface. A pair of angled projections 2160 can also extend upwardly from the charging surface 2154. Angled projections 2160 can interface with opposing curved surfaces of the reservoir of the pump cartridge. Retention projection 2158 and angled projections 2160 are positioned such that they can interface with either pump 102 as shown in FIGS. 1A-1B or pump 202 depicted in FIGS. 2A-2B. The same features are therefore able to retain pumps of different sizes. In addition, retention projection 2158 will abut an end surface of a drive unit of the pump (having a shape matching that of the outline shown on charging surface 2154) if only the drive unit is placed on the charging surface 2154, which represents a third different configuration that can be retained on a charging device by cover 2150.

Cover 2150 can also include a downwardly extending rim 2153 and one or more clips 2156 for retaining cover 2150 on an inductive charging device. In an embodiment there are two clips similar to clips 354 and 356 in FIGS. 4A-4F, but cover 2150 can employ one or more clips of any other configuration.

A variety of features have been described with respect to the various covers disclosed herein. It should be understood that features of each device are not exclusive to that device and that various features from each device can be combined with various features of one or more of the other devices in various other embodiments. Further, the various features can be modified in size, shape, and location to accommodate infusion pumps and other medical devices having various other sizes and shapes to those disclosed herein.

In an embodiment, a cover for an inductive charging device for an ambulatory infusion pump includes an upper surface having a cutout therethrough, the cutout defining an inner perimeter having a shape generally matching a shape of an outer perimeter of an ambulatory infusion pump. A rim can extend downwardly from the upper surface and be configured to engage an outer perimeter of an inductive charging device. A clip can extend downwardly from the rim and be configured to releasably engage with the inductive charging device.

In some embodiments, the cover further comprises a ledge extending inwardly from and around the cutout at a base of the cutout, the ledge defining an inner perimeter of a smaller size that the inner perimeter defined by cutout.

In some embodiments, the clip comprises one or more anti-rotation projections configured to be inserted into an aperture in the inductive charging device to prevent the cover from rotating on the inductive charging device.

In some embodiments, the clip comprises a flexible projection configured to conform to outer perimeter of the inductive charging device.

In some embodiments, the flexible projection is configured to be deformed to fit over the inductive charging device and provide a snap fit with the inductive charging device.

In some embodiments, the clip is a first clip and the cover further comprises a second clip extending downwardly from the rim and configured to releasably engage the cover with the inductive charging device.

In some embodiments, the first clip had a different geometry from the second clip.

In some embodiments, the first clip provides a different function from the second clip.

In some embodiments, the first clip is configured to prevent the cover from rotating on the inductive charging device and the second clip is configured to prevent the cover from being inadvertently dislodged vertically off of the inductive charging device.

In some embodiments, the first clip and the second clip are disposed on opposing sides of the top surface.

In some embodiments, the cover further comprises one or more visual guide features configured to guide a user in optimally orienting the ambulatory infusion pump relative to the cover.

In some embodiments, the cover further comprises one or more visual guide features to guide a user in optimally orienting the cover relating to the inductive charging device.

In some embodiments, the cover further comprises one or more angled projections on an underside of the upper surface configured to interface with a curved surface of the inductive charging device.

An ambulatory infusion pump system including an ambulatory infusion pump including a rechargeable battery and an inductive charging device configured to recharge the rechargeable battery of the ambulatory infusion pump when the ambulatory infusion pump is placed on the inductive charging device. A cover can be selectively attachable to the inductive charging device. The cover can include an upper surface having a cutout therethrough, the cutout defining an inner perimeter having a shape generally matching a shape of an outer perimeter of the ambulatory infusion pump. A rim can extend downwardly from the upper surface and be configured to engage an outer perimeter of the inductive charging device. A clip can extend downwardly from the rim and be configured to releasably engage with the inductive charging device.

In some embodiments, the ambulatory infusion pump system of further comprises a ledge extending inwardly from and around the cutout at a base of the cutout, the ledge defining an inner perimeter of a smaller size that the inner perimeter defined by cutout.

In some embodiments, the ambulatory infusion pump system further comprises a pump cover configured to be inserted over the ambulatory infusion pump, wherein the inner perimeter defined by the cutout has a size generally matching a size of the ambulatory infusion pump when the cover is on the ambulatory infusion pump and the inner perimeter of the ledge has a size generally matching a size of the ambulatory infusion pump without the cover.

In some embodiments, the inductive charging device includes a recess in the outer perimeter of the inductive charging device and the clip comprises one or more anti-rotation projections configured to be inserted into the recess to prevent the cover from rotating on the inductive charging device.

In some embodiments, the clip comprises a flexible projection configured to be deformed to fit over the inductive charging device and provide a snap fit with the inductive charging device.

In some embodiments, the clip is a first clip and the cover further comprises a second clip extending downwardly from the rim and configured to releasably engage the cover with the inductive charging device.

In some embodiments, the first clip has a different geometry from the second clip in order to provide a different function, the first clip being configured to prevent the cover from rotating on the inductive charging device and the second clip being configured to prevent the cover from being inadvertently dislodged vertically off of the inductive charging device.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; 11,911,595; 12,138,425; and 12,214,159 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545; 2023/0113755; 2023/0166033; 2023/0166037; 2023/0173170; 2023/0201452; 2023/0241314; 2023/0277765; 2023/0338653; 2023/0381406; 2024/0050650; 2024/0226423; 2024/0226424 and 2024/0277924; 2024/0399051; 2024/408303; 2024/0416032; 2024/0416033; 2025/0099674; 2025/0099675 2025/0099678; 2025/0099679; and 2025/0108162 and commonly owned U.S. patent application Ser. Nos. 17/368,968; 17/896,492; 18/398,543; 18/962,169; 19/003,140; 19/003,164 and 19/119,554.

For purposes of interpreting the claims, it is expressly intended that the provisions of 35 U.S.C. § 112(f) are not to be invoked unless the specific terms “means for” or “step for” are recited in a claim.

1. A cover for an inductive charging device for an ambulatory infusion pump, comprising:
an upper surface having a cutout therethrough, the cutout defining an inner perimeter having a shape generally matching a shape of an outer perimeter of an ambulatory infusion pump;
a rim extending downwardly from the upper surface configured to engage an outer perimeter of an inductive charging device; and
a clip extending downwardly from the rim and configured to releasably engage with the inductive charging device.

2. The cover of claim 1, further comprising a ledge extending inwardly from and around the cutout at a base of the cutout, the ledge defining an inner perimeter of a smaller size that the inner perimeter defined by cutout.

3. The cover of claim 1, wherein the clip comprises one or more anti-rotation projections configured to be inserted into an aperture in the inductive charging device to prevent the cover from rotating on the inductive charging device.

4. The cover of claim 1, wherein the clip comprises a flexible projection configured to conform to outer perimeter of the inductive charging device.

5. The cover of claim 4, wherein the flexible projection is configured to be deformed to fit over the inductive charging device and provide a snap fit with the inductive charging device.

6. The cover of claim 1, wherein the clip is a first clip and further comprising a second clip extending downwardly from the rim and configured to releasably engage the cover with the inductive charging device.

7. The cover of claim 6, wherein the first clip had a different geometry from the second clip.

8. The cover of claim 7, wherein the first clip provides a different function from the second clip.

9. The cover of claim 8, wherein the first clip is configured to prevent the cover from rotating on the inductive charging device and the second clip is configured to prevent the cover from being inadvertently dislodged vertically off of the inductive charging device.

10. The cover of claim 6, wherein the first clip and the second clip are disposed on opposing sides of the top surface.

11. The cover of claim 1, further comprising one or more visual guide features configured to guide a user in optimally orienting the ambulatory infusion pump relative to the cover.

12. The cover of claim 1, further comprising one or more visual guide features to guide a user in optimally orienting the cover relating to the inductive charging device.

13. The cover of claim 1, further comprising one or more angled projections on an underside of the upper surface configured to interface with a curved surface of the inductive charging device.

14. An ambulatory infusion pump system, comprising:
an ambulatory infusion pump including a rechargeable battery;
an inductive charging device configured to recharge the rechargeable battery of the ambulatory infusion pump when the ambulatory infusion pump is placed on the inductive charging device; and
a cover selectively attachable to the inductive charging device, the cover comprising:
an upper surface having a cutout therethrough, the cutout defining an inner perimeter having a shape generally matching a shape of an outer perimeter of the ambulatory infusion pump;
a rim extending downwardly from the upper surface configured to engage an outer perimeter of the inductive charging device; and
a clip extending downwardly from the rim and configured to releasably engage with the inductive charging device.

15. The ambulatory infusion pump system of claim 14, further comprising a ledge extending inwardly from and around the cutout at a base of the cutout, the ledge defining an inner perimeter of a smaller size that the inner perimeter defined by cutout.

16. The ambulatory infusion pump system of claim 15, further comprising a pump cover configured to be inserted over the ambulatory infusion pump, wherein the inner perimeter defined by the cutout has a size generally matching a size of the ambulatory infusion pump when the cover is on the ambulatory infusion pump and the inner perimeter of the ledge has a size generally matching a size of the ambulatory infusion pump without the cover.

17. The ambulatory infusion pump system of claim 14, wherein the inductive charging device includes a recess in the outer perimeter of the inductive charging device and the clip comprises one or more anti-rotation projections configured to be inserted into the recess to prevent the cover from rotating on the inductive charging device.

18. The ambulatory infusion pump system of claim 14, wherein the clip comprises a flexible projection configured to be deformed to fit over the inductive charging device and provide a snap fit with the inductive charging device.

19. The ambulatory infusion pump system of claim 14, wherein the clip is a first clip and the cover further comprises a second clip extending downwardly from the rim and configured to releasably engage the cover with the inductive charging device.

20. The ambulatory infusion pump system of claim 19, wherein the first clip has a different geometry from the second clip in order to provide a different function, the first clip being configured to prevent the cover from rotating on the inductive charging device and the second clip being configured to prevent the cover from being inadvertently dislodged vertically off of the inductive charging device.
